(19) United States
(12) Patent Application Publication
(10) Pub. No.: US 2026/0216063 A1
(43) Pub. Date: Jul. 30, 2026

(54) METHODS OF SUPPRESSING FREE DNA AND COMPOSITIONS THEREOF

(71) Applicant: PS Therapy, Inc., Westmont, IL (US)

(72) Inventor: Siddharth Narendran, Tamil Nadu (IN)

(21) Appl. No.: 19/147,462

(22) PCT Filed: Jan. 12, 2024
(86) PCT No.: PCT/US2024/011341

§ 371 Date: Jul. 11, 2025

(30) Foreign Application Priority Data
Jan. 13, 2023 (IN) 202341002711

(51) Int. Cl. A61K 9/00; A61K 31/4709; A61K 31/506; A61K 47/02; A61K 47/10; A61K 47/12; A61K 47/18; A61K 47/26; A61K 47/38; A61K 47/40; A61P 29/00

(52) U.S. Cl. CPC A61K 9/0048; A61K 31/4709; A61K 31/506; A61K 47/02; A61K 47/10; A61K 47/12; A61K 47/183; A61K 47/26; A61K 47/38; A61K 47/40; A61P 29/00

(57) ABSTRACT

The present invention provides methods of suppressing, sequestering and/or removing free DNA in and from the eye of a subject comprising topically administering a composition of the present invention to the eye of the subject in need thereof. The present invention is further directed to ophthalmic compositions that are capable of suppressing, sequestering and/or removing free DNA from the eye.

### FIELD OF THE INVENTION

The present invention provides methods of suppressing, sequestering and/or removing free DNA in and from the eye of a subject comprising topically administering a composition of the present invention to the eye of the subject in need thereof. The present invention is further directed to ophthalmic compositions that are capable of suppressing, sequestering and/or removing free DNA from the eye.

### BACKGROUND OF THE INVENTION

The accumulation of DNA outside of a cell membrane in the eye plays a role in many eye conditions. See, for example, Tibrewal, S., Tear fluid extracellular DNA: diagnostic and therapeutic implications in dry eye disease, Invest. Ophthalmol. Vis. Sci. December 2013, 54(13), 8051-8061 and Zeng et al., Neutrophil extracellular traps (NETs) in ocular diseases: an update, Biomolecules 2022, 12, 1440. This DNA is known as “extracellular DNA”, “free DNA” or “cell free DNA” because it exists in the extracellular region of tissues and is “free” or outside any cell membrane, which will be referred to as “free DNA” throughout this patent application. The accumulation of free DNA is harmful as it results in inflammation of the eye. This inflammation can lead to necrosis of vital cells of the eye including retinal cells. The loss of retinal cells can lead to partial or full blindness. To date there is no known treatment for the accumulation of free DNA in the eye.

Thus, there is a need in the art for methods of suppressing, sequestering and/or removing free DNA from the eye of a subject.

### SUMMARY OF THE INVENTION

In certain embodiments, the present invention is directed to ophthalmic compositions capable of suppressing, sequestering and/or removing free DNA in and from the eye.

In certain other embodiments, the present invention is directed to methods of suppressing, sequestering and/or removing free DNA from the eye of a subject comprising topically administering a composition of the present invention to the eye of a subject in need thereof.

In certain other embodiments, the present invention is directed to methods of treating eye conditions comprising topically administering a composition of the present invention to the eye of a subject in need thereof, wherein the eye condition is caused by or is associated with free DNA.

In a preferred embodiment, compositions of the present invention comprise one or more nonionic surfactants, one or more viscosity enhancers, one or more cyclodextrins and water.

In a more preferred embodiment, compositions of the present invention my further comprise one or more additional excipients selected from the group consisting of a polyol, a tonicity adjustor, a disaccharide, a terpenoid, a buffer, a preservative and an antioxidant.

### BRIEF DESCRIPTION OF THE DRAWINGS

FIG. 1. Free DNA binding efficiency of Composition A from Table 1, below, represented as a percentage of free DNA bound.

FIG. 2. TNF-alpha protein levels induced by introduction of A. flavus DNA into human corneal epithelial cells with and without introduction of Composition A from Table 1, below.

FIG. 3. TNF-alpha protein levels induced by introduction of P. aeruginosa DNA into human corneal epithelial cells with and without introduction of Composition A from Table 1, below.

### DETAILED DESCRIPTION OF THE INVENTION

Applicant has surprisingly discovered that compositions of the present invention can suppress the presence of free DNA in the eye. Not to be held to a particular theory, the compositions of the present invention can suppress the presence of free DNA in the eye by sequestering and removing free DNA from the eye. As detailed, above, many eye conditions are cause by and or associated with free DNA. These conditions include, but are not limited to, keratitis including keratitis resulting from bacterial, fungal, or viral infections and DNA-induced inflammation.

As used herein, the term “composition” is intended to encompass a product comprising the specified ingredients in the specified amounts, as well as any product which results, directly or indirectly, from a combination of the specified ingredients in the specified amounts.

As used herein, the term “free DNA” refers to strands of deoxyribonucleic acid that exist outside of a cell membrane. These strands may be single or paired with another strand of deoxyribonucleic acid. In a preferred embodiment “free DNA” may refer to strands of DNA that contain less than about 5 million nucleotides or less than about 5 million base pairs of nucleotides.

As used herein the terms “suppress”, “suppression” and “suppressing” refer to reducing the amount or severity of the object.

As used herein the terms “treat” or “treating” refer to reversing, alleviating, or slowing the progress of the disease, disorder, or condition to which such terms apply, or one or more symptoms of such disease, disorder, or condition.

As used herein, all numerical values relating to amounts, weights, and the like, that are defined as “about” each particular value is plus or minus 10%. For example, the phrase “about 5% w/w” is to be understood as “4.5% to 5.5% w/w.” Therefore, amounts within 10% of the claimed value are encompassed by the scope of the claims.

As used herein “% w/w” refers to the percent weight of the total composition.

As used herein the term “subject” refers but is not limited to a person or other animal.

Throughout the application, the singular forms “a,” “an,” and “the” include plural reference unless the context clearly dictates otherwise.

As used herein the term “polyol” refers to compounds with multiple hydroxyl functional groups available for organic reactions such as monomeric polyols such as glycerin, pentaerythritol, ethylene glycol and sucrose. Further, polyols may refer to polymeric polyols including glycerin, pentaerythritol, ethylene glycol and sucrose reacted with propylene oxide or ethylene oxide.

In certain embodiments, the present invention is directed to ophthalmic compositions capable of suppressing, sequestering and/or removing free DNA in and from the eye.

In another embodiment, the present invention is directed to methods of suppressing, sequestering and/or removing free DNA from the eye of a subject comprising topically administering a composition of the present invention to the eye of a subject in need thereof.

In certain other embodiments, the present invention is directed to methods of treating eye conditions comprising topically administering a composition of the present invention to the eye of a subject in need thereof, wherein the eye condition is caused by or is associated with free DNA. In a preferred embodiment the eye condition to be treated is selected from bacterial keratitis, fungal keratitis, viral keratitis and DNA-induced inflammation. In a more preferred embodiment, the DNA-induced inflammation is caused by an infection, preferably a fungal infection and more preferably an Aspergillus flavus and/or Pseudomonas aeruginosa infection.

In another embodiment, the present invention is directed to a method of treating dry eye caused by or associated with free DNA.

In a preferred embodiment, compositions of the present invention comprise one or more nonionic surfactants, one or more viscosity enhancers, one or more cyclodextrins and water.

In a more preferred embodiment, compositions of the present invention my further comprise one or more additional excipients selected from the group consisting of a polyol, a tonicity adjustor, a disaccharide, a terpenoid, a buffer, a preservative and an antioxidant.

In another preferred embodiment, compositions of the present invention comprise an active ingredient. In a preferred embodiment, the active ingredient is an ophthalmological drug. In a more preferred embodiment, the active ingredient is selected from the group consisting of voriconazole, moxifloxacin, ofloxaxin, levofloxacin, natamycin, olopatadine, lifitegrast, diquafosol, perfluorohexyloctane, timolol, bimatoprost, travoprost, latanoprost, brimonidine, brinzolamide and rho kinase inhibitors and salts and hydrates thereof. Rho kinase inhibitors suitable for use in the present invention include, but are not limited to, fasudil, ripasudil, netarsudil, Y-27632, H-1152, Wf-536, Y-39983, AMA-0076, GSK-269962A, SB-772077-B, SAR-407899 and RKI-1447 and salts and hydrates thereof. In an even more preferred embodiment, the active ingredient is selected from the group consisting of voriconazole and moxifloxacin.

Active ingredients present in compositions of the present invention can be used in the form of salt derived from inorganic or organic acids or bases. Acid addition salts of the active ingredients of the present invention can be prepared in situ during the final isolation and purification of the compounds of the invention or separately by reacting a free base function with a suitable organic acid. Representative acid addition salts include, but are not limited to acetate, adipate, alginate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form acid addition salts include such inorganic acids as hydrochloric acid, hydrobromic acid, hyaluronic acid, and phosphoric acid and such organic acids as oxalic acid, maleic acid, methanosulfonic acid, and succinic acid. Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

Nonionic surfactants suitable for use in the present invention include, but are not limited to, poloxamers, polysorbates, alkylaryl polyethers, polyoxyethyleneglycol alkyl ethers, tyloxapol, and polyoxyls. Poloxamers are nonionic triblock copolymers composed of a central hydrophobic chain of polyoxypropylene (poly(propylene oxide)) flanked by two hydrophilic chains of polyoxyethylene (poly(ethylene oxide)). Polysorbates are oily liquids derived from ethoxylated sorbitan esterified with fatty acids. Polyols are not included in the term “nonionic surfactants.”

Nonionic surfactants may be present in compositions of the present invention at a total concentration from about 0.1% to about 10% w/w, more preferably from about 0.5% to about 5% w/w, even more preferably from about 0.84% to about 2.45% w/w and most preferably at about 0.84%, 1.20%, 1.72% or about 2.45% w/w.

Polysorbates suitable for use in compositions of the present invention include, but are not limited to, polysorbate 20, 60 and 80 and combinations thereof. In a preferred embodiment the polysorbate is polysorbate 80.

Polysorbates may be present in compositions of the present invention at a concentration from about 0.1% to about 5% w/w, preferably from about 0.5% to about 2% w/w, more preferably from about 0.5% to about 1% w/w and most preferably at about 0.5%, 0.7%, or 1% w/w.

Poloxamers suitable for use in compositions of the present invention include, but are not limited to, poloxamer 188, poloxamer 407 and combinations thereof.

Poloxamers may be present in compositions of the present invention at a concentration from about 0.1% to about 5% w/w, preferably from about 0.1% to about 2% w/w, more preferably from about 0.1% to about 1.2% w/w and most preferably at about 0.1%, 0.14%, 0.2%, 0.5%, 0.6%, 0.7%, 0.84%, 1.0% or 1.2% w/w.

Polyoxyls suitable for use in the present invention include, but are not limited to, polyoxyl castor oils including, but not limited to, polyoxyl 30 castor oil, polyoxyl 35 castor oil and combinations thereof.

Polyoxyls may be present in compositions of the present invention at a concentration from about 0.05% to about 1% w/w, preferably from about 0.1% to about 0.5% w/w, more preferably from about 0.18% to about 0.25% w/w and most preferably at about 0.18% or about 0.25% w/w.

Viscosity enhancers suitable for use in the present invention include, but are not limited to, cellulose derivatives, carbomers, gums, dextrans, dendrimers, polyvinyl alcohols (“PVA”), polyvinyl pyrrolidones (“PVP”, povidones), polyacrylic acids, polyethylene glycols, propylene glycol, chitosans, hyaluronates, hyaluronic acids and combinations thereof.

Viscosity enhancers may be present in compositions of the present invention at a concentration from about 0.01% to about 10% w/w, preferably from about 0.01% to about 5% w/w, even more preferably from about 0.04% to about 4% w/w and most preferably at about 0.04%, 0.5%, 0.7%, 0.74%, 0.75%, 1%, 1.24%, 1.5%, 1.7%, 1.74%, 1.75%, 2%, 2.54% or 4% w/w.

Cellulose derivatives suitable for use in the present invention include, but are not limited to, carboxymethyl cellulose (“CMC”), CMC high molecular weight blend, CMC low molecular weight blend, CMC moderate molecular weight blend, methylcellulose, methyl cellulose 4000, hydroxymethyl cellulose, hydroxypropyl cellulose, hydroxypropylmethyl cellulose (“HPMC”), HPMC high molecular weight blend, hydroxylpropylmethyl cellulose 2906, carboxypropylmethyl cellulose (“CPMC”), CPMC high molecular weight blend, hydroxyethyl cellulose, hydroxymethyl cellulose and combinations thereof.

In a preferred embodiment, polyethylene glycols may have a molecular weight from about 200 Daltons to about 20,000 Daltons. In a more preferred embodiment, the polyethylene glycols have a molecular weight from about 380 to about 420 Daltons and most preferably about 400 Daltons.

In another preferred embodiment, polyvinyl alcohols and polyvinyl pyrrolidones may have a molecular weight from about 1 to about 1,000 kilodaltons. In a more preferred embodiment, the polyvinyl alcohols and polyvinyl pyrrolidones have a molecular weight from about 1 to about 500 kilodaltons and most preferably about 40 kilodaltons or about 360 kilodaltons.

Cyclodextrins are composed of 5 or more α-D-glucopyranoside units linked together at position 1 and 4. Cyclodextrins suitable for use in the present invention include, but are not limited to, alpha, beta and gamma cyclodextrins and combinations thereof.

Cyclodextrins may be present in compositions of the present invention at a concentration from about 1% to about 5% w/w, more preferably from about 1% to about 2% w/w, even more preferably from about 1.05% to about 2% w/w and most preferably at about 1.05%, 1.5% or about 2% w/w.

Beta-cyclodextrins suitable for use in the present invention include, but are not limited to, hydroxypropyl beta-cyclodextrin, sulfobutylether β-cyclodextrin and combinations thereof.

Gamma-cyclodextrins suitable for use in the present invention include, but are not limited to, hydroxypropyl gamma-cyclodextrin.

Polyols suitable for use in the subject invention include, but are not limited to, mannitol, xylitol, sorbitol, isosorbide, erythritol, glycerol, maltitol and combinations thereof.

Polyols may be present in compositions of the present invention at a concentration from about 0.1% to about 10% w/w, preferably from about 0.2% to about 7% w/w, even more preferably from about 0.35% to about 5% w/w and most preferably at about 0.35%, 0.53% or about 5% w/w.

Tonicity adjustors suitable for use in the present invention include, but are not limited to, magnesium chloride, sodium chloride, potassium chloride and combinations thereof.

Tonicity adjustors may be present in compositions of the present invention at a concentration from 0.01% to about 2% w/w, preferably from about 0.05% to about 1% w/w, even more preferably from about 0.07% to about 0.65% w/w and most preferably at about 0.07%, 0.25%, 0.32%, 0.35%, 0.42%, 0.55%, 0.62% or 0.65% w/w.

Disaccharides suitable for use in the present invention include, but are not limited to, non-reducing disaccharides such as trehalose.

Disaccharides may be present in compositions of the present invention at a concentration from 0.1% to about 5% w/w, preferably from about 0.5% to about 2% w/w, even more preferably from about 1% to about 1.5% w/w and most preferably at about 1% or 1.5% w/w.

Terpenoids suitable for use in the present invention include, but are not limited to, citral, WS-12, icilin and menthol. In a preferred embodiment, the terpenoid is menthol.

Terpenoid may be present in compositions of the present invention at a concentration from about 0.01 to about 4.00 mM, more preferably from about 0.01 to about 2.0 mM, even more preferably from about 0.1 to about 1.0 mM, yet even more preferably from about 0.35 to about 0.6 mM, and most preferably at about 0.01, 0.07, 0.1, 0.14, 0.15, 0.2, 0.27, 0.30, 0.32, 0.34, 0.35, 0.36, 0.37, 0.38, 0.40, 0.42, 0.44, 0.46, 0.48, 0.5, 0.6, 0.65, 0.7, 0.75, 0.8, 0.85, 1.0, 1.2, 1.5, 1.6, 1.75, 2.0 or 4.0 mM.

Buffers and pH adjustors suitable for use in the present invention include, but are not limited to, acetate buffers, carbonate buffers, citrate buffers, phosphate buffers and borate buffers. In a preferred embodiment, the buffers and pH adjustors are at a concentration from about 1 to about 100 millimolar, more preferably from about 3 to about 10 millimolar and most preferably about 3, 4, 5, 5.5, 6, 6.5, 7, 7.5, 8, 8.5, 9, 9.5 or 10 millimolar It is understood that various acids or bases can be used to adjust the pH of the composition as needed. pH adjusting agents include, but are not limited to, sodium hydroxide and hydrochloric acid. pH of the compositions can be from 4.0 to 8.0, more preferably from about 5.0 to about 8.0 and from about 5.0 to about 6.0, and most preferably about 6.0.

Preservatives suitable for use in the present invention include, but are not limited to, benzalkonium chloride (“BAK”), sorbate, methylparaben, polypropylparaben, chlorobutanol, thimerosal, phenylmercuric acetate, perborate, phenylmercuric nitrate and combinations thereof. In a preferred embodiment, the preservative is potassium sorbate.

Preservatives may be present in compositions of the present invention at a concentration from about 0.01% to about 1% w/w, preferably from about 0.05% to about 0.5% w/w, even more preferably from about 0.1% to about 0.12% w/w and most preferably at about 0.1% w/w.

Antioxidants suitable for use in the present invention include, but are not limited to, citrate. EDTA, disodium EDTA, sodium metabisulfite, sodium thiosulfate, acetylcysteine, butylated hydroxyanisole and butylated hydroxytoluene and a combination thereof. In a preferred embodiment, the antioxidant is disodium EDTA.

Antioxidants may be present in compositions of the present invention at a concentration from about 0.01% to about 1% w/w, preferably from about 0.05% to about 0.5% w/w, even more preferably from about 0.1% to about 0.12% w/w and most preferably at about 0.1% w/w.

In a preferred embodiment, the present invention is directed to a method of suppressing free DNA in the eye of a subject in need thereof comprising topically administering to the subject in need thereof a composition comprising:
one or more nonionic surfactants, preferably selected from the group consisting of poloxamers, polysorbates, alkylaryl polyethers, polyoxyethyleneglycol alkyl ethers, tyloxapol, and polyoxyls, more preferably selected from the group consisting of polysorbate 20, poloxamer 188, poloxamer 407 and polyoxyl castor oil;
one or more viscosity enhancers, preferably selected the group consisting of cellulose derivatives, carbomers, gums, dextrans, dendrimers, polyvinyl alcohols, polyvinyl pyrrolidones, polyacrylic acids, polyethylene glycols, propylene glycol, chitosans, hyaluronates and hyaluronic acids, more preferably selected from the group consisting of cellulose derivatives, dendrimers, polyvinyl alcohols and polyvinyl pyrrolidones;
one or more cyclodextrins, preferably selected from the group consisting of hydroxypropyl beta-cyclodextrin and hydroxypropyl gamma-cyclodextrin; and
water; and
optionally, one or more additional excipients selected from the group consisting of:
a polyol selected from the group consisting of mannitol, xylitol, sorbitol, isosorbide, erythritol, glycerol, maltitol and combinations thereof, preferably mannitol;
a tonicity adjustor selected from the group consisting of magnesium chloride, sodium chloride, potassium chloride, and combinations thereof;
a disaccharide;
a terpenoid selected from the group consisting of citral, WS-12, icilin, menthol, and combinations thereof, preferably menthol;
a buffer selected from the group consisting of acetate buffers, carbonate buffers, citrate buffers, phosphate buffers, borate buffers, and combinations thereof, preferably citrate buffer;
a preservative selected from the group consisting of benzalkonium chloride, sorbate, methylparaben, polypropylparaben, chlorobutanol, thimerosal, phenylmercuric acetate, perborate, phenylmercuric nitrate, and combinations thereof, preferably potassium sorbate; and
an antioxidant selected from the group consisting of citrate, EDTA, disodium EDTA, sodium metabisulfite, sodium thiosulfate, acetylcysteine, butylated hydroxyanisole and butylated hydroxytoluene and combinations thereof, preferably disodium EDTA.

The disclosed embodiments are simply embodiments of the inventive concepts disclosed herein and should not be considered as limiting unless the claims expressly state otherwise.

The following examples are intended to illustrate the present invention and to teach one of ordinary skill in the art how to use the formulations of the invention. They are not intended to be limiting in any way.

### EXAMPLE

### Example 1—Free DNA Suppression (Hypothetical)

TABLE 1

| Composition | A | B | C | #1 | #2 |
|---|---|---|---|---|---|
| Polysorbate 80 | 0.70% | 1.00% | 1.00% | — | — |
| Poloxamer 407 | 0.70% | 1.00% | 1.00% | 0.70% | 1.0% |
| Poloxamer 188 | 0.14% | 0.20% | 0.20% | 0.14% | 0.20% |
| Povidone K90 | — | — | — | 1.5% | 2.0% |
| Polyoxyl Castor oil | 0.18% | 0.25% | 0.25% | — | — |
| PEG-400 | 0.70% | 0.75% | 0.75% | 1.0% | 1.5% |
| Mannitol | 0.53% | 0.35% | 0.35% | 5.0% | 5.0% |
| Magnesium Chloride | 0.07% | — | — | — | — |
| Sodium Chloride | 0.55% | 0.55% | 0.55% | — | — |
| Citrate Buffer | 4 mM | 4 mM | 4 mM | 4 mM | 4 mM |
| Hydroxypropyl | 0.04% | 1.00% | 1.00% | 0.04% | 0.50% |
| methylcellulose | | | | | |
| Potassium Sorbate | 0.10% | 0.10% | 0.10% | 0.10% | 0.10% |
| Di Sodium EDTA | 0.10% | 0.10% | 0.10% | — | — |
| Hydroxypropyl | 1.05% | 1.50% | 1.50% | 1.5% | 2.0% |
| gamma cyclodextrin | | | | | |
| Hydroxypropyl beta | — | — | — | — | — |
| cyclodextrin | | | | | |
| Dendrimer | — | — | — | — | — |
| PVP 40 | — | — | — | — | — |
| PVA 40 | — | — | — | — | — |
| Menthol | — | 0.35 mM | 0.60 mM | — | — |
| Water for Injection | QS | QS | QS | QS | QS |
| (WFI) | | | | | |
| pH | 6.0 | 6.0 | 6.0 | 6.0 | 6.0 |
| Composition | #3 | #4 | #5 | #6 | #7 |
| Polysorbate 80 | — | 0.70% | 0.70% | 0.50% | 0.70% |
| Poloxamer 407 | 1.0% | 0.70% | 0.70% | 0.50% | 0.70% |
| Poloxamer 188 | 0.20% | 0.14% | 0.14% | 0.10% | 0.14% |
| Povidone K90 | 2.0% | 0.50% | — | — | — |
| Polyoxyl Castor oil | — | 0.18% | 0.18% | 0.10% | 0.18% |
| PEG-400 | 1.5% | 0.70% | 0.70% | 0.70% | 0.70% |
| Mannitol | 5.0% | 0.53% | — | 0.53% | 0.53% |
| Magnesium Chloride | — | 0.07% | — | 0.07% | 0.07% |
| Sodium Chloride | — | 0.35% | 0.65% | 0.25% | 0.55% |
| Citrate Buffer | 4 mM | 4 mM | 4 mM | 4 mM | 4 mM |
| Hydroxypropyl | 0.50% | 0.04% | 0.04% | — | 0.04% |
| methylcellulose | | | | | |
| Potassium Sorbate | 0.10% | 0.10% | 0.10% | 0.10% | 0.10% |
| Di Sodium EDTA | — | 0.10% | 0.10% | 0.10% | 0.10% |
| Hydroxypropyl | — | 1.05% | 1.05% | 1.05% | 1.05% |
| gamma-cyclodextrin | | | | | |
| Hydroxypropyl beta- | 2.0% | — | — | — | — |
| cyclodextrin | | | | | |
| Dendrimer | — | — | — | — | 1.00% |
| PVP 40 | — | — | — | 0.50% | — |
| PVA 40 | — | — | — | 0.50% | — |
| Water for Injection | QS | QS | QS | QS | QS |
| (WFI) | | | | | |
| pH | 6.0 | 6.0 | 6.0 | 6.0 | 6.0 |

### Method

The compositions from Table 1, above, were each topically administered to the eye of a subject previously diagnosed with the presence of free DNA in the eye. Free DNA concentration was measured both before and after administration.

### Results

Following administration of each composition of Table 1, above, the presence of free DNA in the eye was significantly reduced.

### Example 2—Free DNA Suppression

### Method

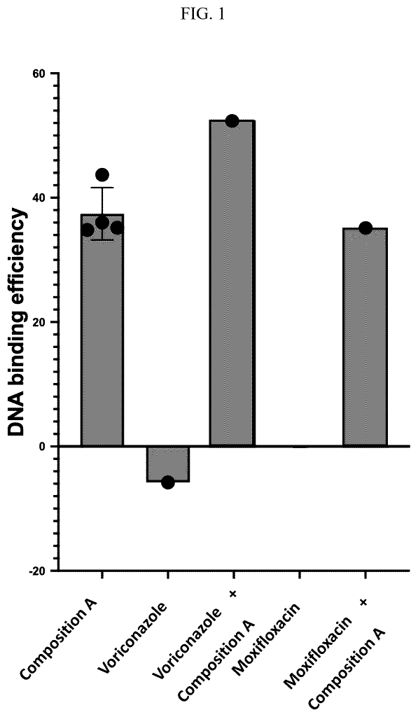

Voriconazole, moxifloxacin, composition A from Table 1, above, a combination of voriconazole and composition A and a combination of moxifloxacin and composition A were each individually incubated with free DNA in a petri dish with and without the addition of voriconazole or moxifloxacin. The percentage of free DNA bound was measured using an ethidium bromide displacement assay. See results in FIG. 1.

### Results

0% of free DNA was bound by voriconazole or moxifloxacin alone. 37% of free DNA was bound by composition A. The mixture of voriconazole and composition A bound 52% of free DNA. The mixture of moxifloxacin and composition A bound 35% of free DNA. See FIG. 1. Thus, compositions of the present invention are capable of binding and/or sequestering free DNA.

### Example 3—Free DNA Suppression

### Method

Compositions #1-3 from Table 1, above, were each individually incubated with free DNA in a petri dish at concentrations of 1% and 10% in PBS buffer. The percentage of free DNA bound was measured using an ethidium bromide displacement assay. See Table 2, below, for results.

TABLE 2

| Composition | DNA binding efficiency |
|---|---|
| Composition #1 @ 10% | 9.8% |
| Composition #1 @ 1% | 13.7% |
| Composition #2 @ 10% | 11.2% |
| Composition #2 @ 1% | 18.8% |
| Composition #3 @ 10% | 13.1% |
| Composition #3 @ 1% | 15.4% |

### Results

9.8% of free DNA was bound by composition #1 at 10% concentration and 13.7% was bound at 1%. 11.2% of free DNA was bound by composition #2 at 10% concentration and 18.8% was bound at 1%. 13.1% of free DNA was bound by composition #3 at 10% concentration and 15.4% was bound at 1%. Thus, compositions of the present invention are capable of binding and/or sequestering free DNA.

### Example 4—Treatment of DNA-Induced Inflammation Method

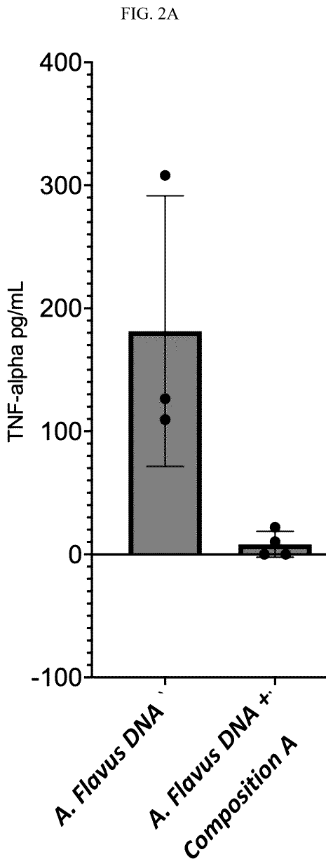

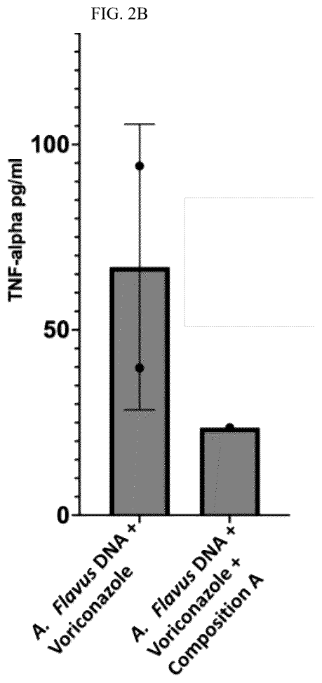

Tumor Necrosis Factor alpha secretion (“TNF-alpha”) is a marker for DNA-induced inflammation. See, Campbell et al., CpG-containing immunostimulatory DNA sequences elicit TNF-alpha-dependent toxicity in rodents but not in humans, 2009, J Clin Invest, 119(9), 2564-2576. 3 sets of differentiated human leukemia monocytic (“differentiated THP-1”) cells were infected with 6 micrograms of A. flavus DNA, 6 micrograms of A. flavus DNA and composition A, 6 micrograms of A. flavus DNA and voriconazole, or 6 micrograms of A. flavus DNA, voriconazole and composition A. 24 hours post infection the cells were assayed for TNF-alpha protein levels via enzyme-linked immunosorbent assays (“ELISA”). Results of this study can be found in FIGS. 2A and 2B.

### Results

Administration of composition A from Table 1, above, reduced TNF-alpha protein levels from 180 picograms per milliliter to 10 picograms per milliliter, see FIG. 2A, below. The addition of composition A from Table 1, above, to voriconazole reduced TNF-alpha protein levels from 85 picograms per milliliter to 45 picograms per milliliter, see FIG. 2B, below. Thus, compositions of the present invention are capable of treating inflammation caused by free DNA.

### Example 5—Treatment of DNA-Induced Inflammation

### Method

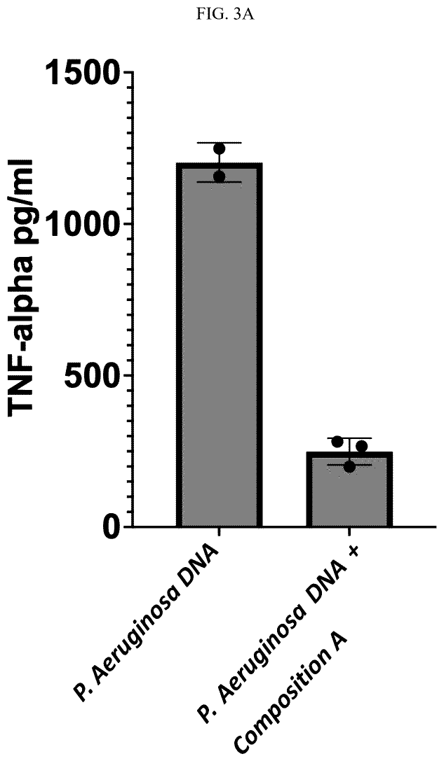

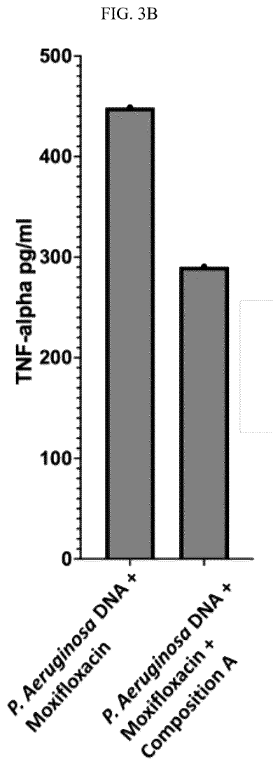

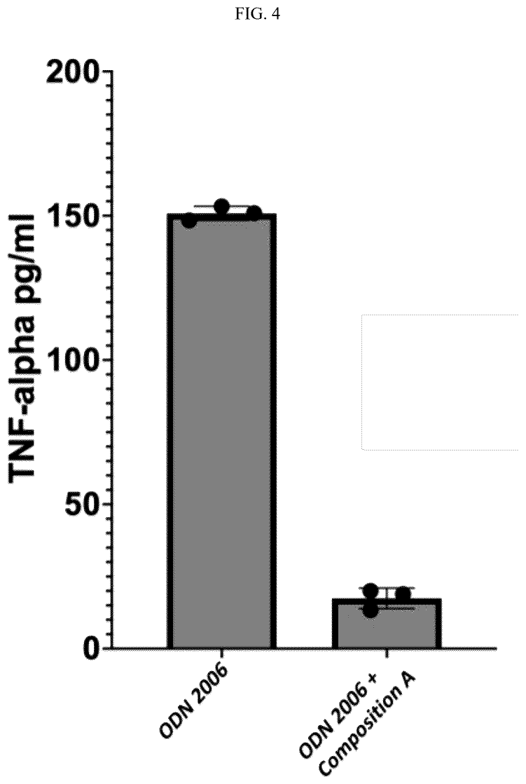

3 sets of differentiated THP-1 cells were infected with 6 micrograms of P. aeruginosa, 6 micrograms of P. aeruginosa DNA and composition A from Table 1, above, 6 micrograms of P. aeruginosa DNA and moxifloxacin, or 6 micrograms of P. aeruginosa DNA, moxifloxacin and composition A. 24 hours post infection the cells were assayed for TNF-alpha protein levels via ELISA. Results of this study can be found in FIGS. 3A and 3B.

### Results

Administration of composition A from Table 1, above, reduced TNF-alpha protein levels from 1,200 picograms per milliliter to 250 picograms per milliliter, see FIG. 3A, below. The addition of composition A from Table 1, above, to moxifloxacin reduced TNF-alpha protein levels from 450 picograms per milliliter to 285 picograms per milliliter, see FIG. 3B, below. Thus, compositions of the present invention are capable of treating inflammation caused by free DNA.

### Example 6—Treatment of DNA-Induced Inflammation

### Method

6 sets of differentiated THP-1 cells were infected with 1) nothing, 2) lipofectamine™ 2000 transfectin reagent, 3) 6 micrograms of A. flavus DNA, 4) 6 micrograms of A. flavus DNA and composition #1 from Table 1, above, 5) 6 micrograms of A. flavus DNA and composition #2 from Table 1, above, 6) 6 micrograms of A. flavus DNA and composition #3 from Table 1, above. 24 hours post infection the cells were assayed for TNF-alpha protein levels via ELISA. Results of this study can be found in Table 3, below.

TABLE 3

| | TNF-alpha protein levels |
|---|---|
| Composition | (pg/ml) |
| THP1 cells | 41.4 |
| THP1 cells + lipofectamineTM 2000 | 63.9 |
| THP1 cells + A. flavus DNA | 109.5 |
| THP1 cells + A. flavus DNA + | 24.8 |
| Composition #1 | |
| THP1 cells + A. flavus DNA + | 25.7 |
| Composition #2 | |
| THP1 cells + A. flavus DNA + | 12.1 |
| Composition #3 | |

### Results

Administration of compositions #1-3 from Table 1, above, reduced TNF-alpha protein levels from 109.4 picograms per milliliter to 24.8, 25.7 and 12.1 picograms per milliliter, respectively. See Table 3, above. Thus, compositions of the present invention are capable of treating inflammation caused by free DNA.

### Example 7—Treatment of DNA-Induced Inflammation

### Method

TNF-alpha secretion is a marker for DNA-induced inflammation. 3 sets of differentiated THP-1 cells were infected with 6 micrograms of ODN 2006, a synthetic DNA strand and 6 micrograms of ODN 2006 and composition A from Table 1, above. 24 hours post infection the cells were assayed for TNF-alpha protein levels via ELISA. Results of this study can be found in FIG. 4.

### Results

Administration of composition A from Table 1, above, reduced TNF-alpha protein levels from 150 picograms per milliliter to 15 picograms per milliliter, see FIG. 4. Thus, compositions of the present invention are capable of treating inflammation caused by free DNA.

### Example 7—Treatment of DNA-Induced Inflammation

### Method

TNF-alpha secretion is a marker for DNA-induced inflammation. 3 sets of differentiated THP-1 cells were infected with 1) nothing 2) 6 micrograms of ODN 2006 and lipofectamine™ 2000 or 6 micrograms of ODN 2006, lipofectamine™ 2000 and composition #3 from Table 1, above. 24 hours post infection the cells were assayed for TNF-alpha protein levels via ELISA. Results of this study can be found in Table 4, below.

TABLE 4

| | TNF-alpha protein levels |
|---|---|
| Composition | (pg/ml) |
| THP1 cells | 51.65 |
| THP1 cells + lipofectamine ™ 2000 + | 153.22 |
| ODN2006 | |
| THP1 cells + A. flavus DNA + | 33.38 |
| lipofectamine ™ 2000 + | |
| Composition #3 | |

### Results

Administration of composition #3 from Table 1, above, reduced TNF-alpha protein levels from 153.22 picograms per milliliter to 33.38 picograms per milliliter, see Table 4, above. Thus, compositions of the present invention are capable of treating inflammation caused by free DNA.

What is claimed is:

1. A method of suppressing free DNA in the eye of a subject in need thereof comprising topically administering to the subject in need thereof a composition comprising one or more nonionic surfactants, one or more viscosity enhancers, one or more cyclodextrins and water.

2. The method of claim 1, wherein the one or more nonionic surfactants are selected from the group consisting of poloxamers, polysorbates, alkylaryl polyethers, polyoxyethyleneglycol alkyl ethers, tyloxapol, and polyoxyls.

3. The method of claim 2, wherein the one or more nonionic surfactants are selected from the group consisting of polysorbate 20, poloxamer 188, poloxamer 407 and polyoxyl castor oil.

4. The method of claim 1, wherein the one or more viscosity enhancers are selected from the group consisting of cellulose derivatives, carbomers, gums, dextrans, dendrimers, polyvinyl alcohols, polyvinyl pyrrolidones, polyacrylic acids, polyethylene glycols, propylene glycol, chitosans, hyaluronates and hyaluronic acids.

5. The method of claim 4, wherein the one or more viscosity enhancers are selected from the group consisting of cellulose derivatives, dendrimers, polyvinyl alcohols and polyvinyl pyrrolidones.

6. The method of claim 1, wherein the one or more cyclodextrins are selected from the group consisting of hydroxypropyl beta-cyclodextrin and hydroxypropyl gamma-cyclodextrin.

7. The method of claim 1, wherein the composition further comprises one or more additional excipients selected from the group consisting of a polyol, a tonicity adjustor, a disaccharide, a terpenoid, a buffer, a preservative and an antioxidant.

8. The method of claim 7, wherein the one or more additional excipients are selected from the group consisting of mannitol, sodium chloride, magnesium chloride, menthol, potassium sorbate, disodium EDTA and citrate buffer.

9. The method of claim 1, wherein the composition further comprises an active ingredient.

10. A method of suppressing free DNA in the eye of a subject in need thereof comprising topically administering to the subject in need thereof a composition comprising from about 0.5% to about 5% w/w of one or more nonionic surfactants, from about 0.01% to about 5% w/w of one or more viscosity enhancers, from about 1% to about 2% w/w of one or more cyclodextrins and water, wherein w/w denotes weight by total weight of the composition.

11. The method of claim 10, wherein the composition comprises from about 0.84% to about 2.45% w/w of one or more nonionic surfactants, from about 0.04% to about 4% w/w of one or more viscosity enhancers, from about 1.05% to about 2% w/w of one or more cyclodextrins and water.

12. The method of claim 10, wherein the composition further comprises one or more additional excipients selected from the group consisting of from about 0.2% to about 7% w/w of a polyol, from about 0.05% to about 1% w/w of a tonicity adjustor, from about 0.1% to about 5% w/w of a disaccharide, from about 0.1 to about 1 millimolar of a terpenoid, from about 3 to about 10 millimolar of a buffer, from about 0.05% to about 0.5% w/w of a preservative and from about 0.05% to about 0.5% w/w of an antioxidant.

13. The method of claim 12, wherein the composition comprises one or more additional excipients selected from the group consisting of from about 0.35% to about 5% w/w of a polyol, from about 0.07% to about 0.65% w/w of a tonicity adjustor, from about 0.5% to about 2% w/w of a disaccharide, from about 0.35 to about 0.6 millimolar of a terpenoid, from about 4 millimolar of a buffer, from about 0.1% to about 0.12% w/w of a preservative and from about 0.1% to about 0.12% w/w of an antioxidant.

14. The method of claim 10, wherein the composition further comprises an active ingredient.

15. A method of treating an eye condition comprising topically administering to a subject in need thereof a composition comprising one or more nonionic surfactants, one or more viscosity enhancers, one or more cyclodextrins and water, wherein the eye condition is caused by or is associated with free DNA.

16. The method of claim 15 wherein the eye condition is selected from bacterial keratitis, fungal keratitis, viral keratitis and DNA-induced inflammation.

17. The method of claim 15, wherein the composition further comprises one or more additional excipients selected from the group consisting of a polyol, a tonicity adjustor, a disaccharide, a terpenoid, a buffer, and a preservative.

18. The method of claim 16, wherein the composition further comprises an active ingredient.

19. An ophthalmic composition for suppressing free DNA comprising polysorbate 80, poloxamer 407, poloxamer 188, polyoxyl castor oil, polyethylene glycol 400, mannitol, hydroxypropylmethyl cellulose, hydroxypropyl gamma-cyclodextrin, dendrimer and water.

20. The composition of claim 19, further comprising magnesium chloride, sodium chloride, citrate buffer, potassium sorbate and disodium EDTA.

21. The composition of claim 19, further comprising an active ingredient.

22. The composition of claim 19, comprising:
about 0.7% w/w polysorbate 80;
about 0.7% w/w poloxamer 407;
about 0.14% w/w poloxamer 188;
about 0.18% w/w polyoxyl castor oil;
about 0.7% w/w polyethylene glycol 400;
about 0.07% w/w mannitol;
about 0.04% w/w hydroxypropylmethyl cellulose;
about 1.05% w/w hydroxypropyl gamma-cyclodextrin; and
about 1% w/w dendrimer,
wherein w/w denotes weight by total weight of the composition.
